(19) United States
(12) Patent Application Publication
(10) Pub. No.: US 2025/0295335 A1
(43) Pub. Date: Sep. 25, 2025

(54) MEASURING INSTRUMENT

(71) Applicant: Sharp Semiconductor Innovation Corporation, Tenri City (JP)

(72) Inventors: Qichen WANG, Tenri City (JP); Daisuke FUNAO, Tenri City (JP); Son Nam HOANG, Tenri City (JP); Sota HIDA, Tenri City (JP); Yuta MOCHIZUKI, Tenri City (JP)

(21) Appl. No.: 19/083,651

(22) Filed: Mar. 19, 2025

(30) Foreign Application Priority Data
Mar. 22, 2024 (JP) 2024-046571

(51) Int. Cl. A61B 5/1455

(52) U.S. Cl. CPC A61B 5/14552

(57) ABSTRACT

A measuring instrument includes: a camera that captures an image of a living body at a distance from the living body, the camera including an imaging element including: a plurality of types of first pixels that respectively exhibit a plurality of mutually different first peak sensitivity wavelengths of either less than or equal to 600 nm or greater than or equal to 760 nm; and a second pixel that exhibits a second peak sensitivity wavelength of greater than or equal to 620 nm and less than or equal to 740 nm; and a processing unit that acquires pulse waves from a plurality of first signals respectively representing quantities of light received by the plurality of types of first pixels and also from a second signal representing a quantity of light received by the second pixel and that acquires an oxygen saturation level of the living body from the pulse waves.

### CROSS-REFERENCE TO RELATED APPLICATION

The present application claims priority from Japanese Application JP2024-046571, the content of which is hereby incorporated by reference into this application.

### BACKGROUND OF THE INVENTION

### 1. Field of the Invention

The present disclosure relates to a measuring instrument.

### 2. Description of the Related Art

PCT International Application Publication No. WO2017/085793 discloses an endoscope system. In this endoscope system, narrow-bandwidth light is projected onto a subject. The projected narrow-bandwidth light exhibits a spectrum with peaks in wavelength ranges where R, G, and B pixels have high spectral sensitivity. Meanwhile, image data is acquired from a camera head. In addition, an oxygen saturation level is calculated using G pixel values and R pixel values contained in the image data (paragraphs [0030] to [0032] and [0053] to [0057]).

### SUMMARY OF THE INVENTION

In the endoscope system disclosed in Patent Literature 1, the light projected onto a subject contains no environmental light. Therefore, the calculated oxygen saturation level is not affected by variations of environmental light.

A pulse wave of a living body can be acquired in a contactless manner by capturing images of the living body with a camera to generate signals and then acquiring a pulse wave from the generated signals. However, the pulse wave acquired in a contactless manner may be affected by, for example, body movements of the living body and variations of environmental light. Therefore, the pulse wave acquired in a contactless manner does not have the precision that is sufficient to acquire an oxygen saturation level from this pulse wave.

The present disclosure, in an aspect thereof, has been made in view of this problem. The present disclosure, in an aspect thereof, has an object to provide, for example, a measuring instrument that enables restraining the influence of body movements of the living body and variations of environmental light to acquire an oxygen saturation level in a contactless manner.

The present disclosure, in an aspect thereof, is directed to a measuring instrument including: a camera that captures an image of a living body at a distance from the living body, the camera including an imaging element including: a plurality of types of first pixels that respectively exhibit a plurality of mutually different first peak sensitivity wavelengths of either less than or equal to 600 nm or greater than or equal to 760 nm; and a second pixel that exhibits a second peak sensitivity wavelength of greater than or equal to 620 nm and less than or equal to 740 nm; and a processing unit that acquires a plurality of pulse waves from a plurality of first signals respectively representing quantities of light received by the plurality of types of first pixels and also from a second signal representing a quantity of light received by the second pixel and that acquires an oxygen saturation level of the living body from the plurality of pulse waves.

### BRIEF DESCRIPTION OF THE DRAWINGS

FIG. 1 is a block diagram of a measuring instrument in accordance with Embodiment 1.

FIG. 2 is a schematic cross-sectional view of a camera included in the measuring instrument in accordance with Embodiment 1.

FIG. 3 is a schematic plan view of a plurality of pixels included in the measuring instrument in accordance with Embodiment 1.

FIG. 4 is a graph representing an example of the spectral sensitivities of pixels included in a biological information measuring instrument in accordance with Embodiment 1.

FIG. 5 is a diagram illustrating an oxygen saturation level calculation process performed by a processing unit included in the measuring instrument in accordance with Embodiment 1.

FIG. 6 is a diagram illustrating a pulse wave amplitude calculation process performed by the processing unit included in the measuring instrument in accordance with Embodiment 1.

FIG. 7 is a diagram illustrating an example of the peak sensitivity wavelengths of pixels included in the measuring instrument in accordance with Embodiment 1.

FIG. 8 is a diagram illustrating an example of the peak sensitivity wavelengths of pixels included in the measuring instrument in accordance with Embodiment 1.

FIG. 9 is a diagram illustrating an example of the peak sensitivity wavelengths of pixels included in the measuring instrument in accordance with Embodiment 1.

FIG. 10 is a flow chart representing a flow of a process performed by the processing unit included in the measuring instrument in accordance with Embodiment 1.

FIG. 11 is a schematic illustration of a pulse oximeter for comparison with the measuring instrument in accordance with Embodiment 1.

FIG. 12 is a block diagram of a measuring instrument in accordance with Embodiment 2.

FIG. 13 is a flow chart representing a flow of a process performed by a processing unit included in the measuring instrument in accordance with Embodiment 2.

FIG. 14 is an illustration of an image represented by a plurality of signals outputted by an imaging element included in a measuring instrument in accordance with Embodiment 3 and also of a frame representing an area specified by a processing unit included in this measuring instrument.

### DETAILED DESCRIPTION OF THE INVENTION

The following will describe embodiments of the present disclosure with reference to drawings. Identical and equivalent elements in the drawings are denoted by the same reference numerals, and description thereof is not repeated.

### 1 Embodiment 1

### 1.1 Measuring Instrument

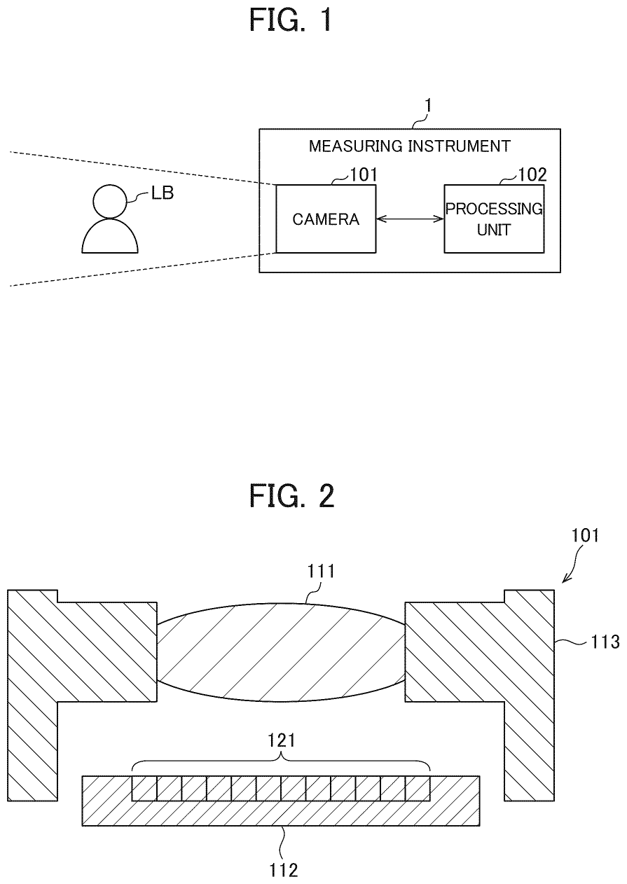

FIG. 1 is a block diagram of a measuring instrument in accordance with Embodiment 1.

A measuring instrument 1 in accordance with Embodiment 1 shown in FIG. 1 measures an oxygen saturation level of a living body LB. In doing so, the measuring instrument 1 generates a signal in accordance with the light reflected off the living body LB by capturing an image of the living body LB, acquires a volume pulse wave of the living body LB from the generated signal, and acquires the oxygen saturation level from the acquired volume pulse wave. Hence, the measuring instrument 1 acquires a volume pulse wave in a contactless manner and measures the oxygen saturation level in a contactless manner.

The living body LB is the body of a living organism. This living organism has a cardiovascular system for circulating blood containing oxidized hemoglobin and reduced hemoglobin. The living organism is, for example, a human.

The acquired volume pulse wave is caused by variations of the intensity of the light reflected off the living body LB, the variations being in turn caused by repetition of alternate expansion and contraction of blood vessels in which blood flows.

The variations of the intensity of the reflected light caused by the repetition of alternate expansion and contraction of blood vessels are as small as approximately a few tenths of a percent. Therefore, volume pulse waves have a small amplitude. Therefore, the volume pulse wave acquired in a contactless manner is generally easily affected by, for example, body movements of the living body LB and variations of environmental light. Therefore, it is generally difficult to acquire an oxygen saturation level with high precision from the volume pulse wave acquired in a contactless manner. The measuring instrument 1 can overcome this problem, thereby acquiring an oxygen saturation level with high precision from a volume pulse wave acquired in a contactless manner.

Referring to FIG. 1, the measuring instrument 1 includes a camera 101 and a processing unit 102.

The camera 101 captures an image of the living body LB at a distance from the living body LB and outputs a signal in accordance with reflected light.

The processing unit 102 controls the camera 101. The processing unit 102 acquires a volume pulse wave from the outputted signal to acquire an oxygen saturation level from the acquired volume pulse wave. The processing unit 102 includes a processor, a memory, and peripheral circuitry. The processor executes a program stored in the memory to cause the processor, the memory, and the peripheral circuitry to function as the processing unit 102. The process performed by the processing unit 102 may be either entirely or partially performed by a dedicated electronic circuit.

An oxygen saturation level can be acquired in a contactless manner by acquiring the volume pulse wave from the signal outputted by the camera 101, which captures an image of the living body LB at a distance from the living body LB, and acquiring the oxygen saturation level from the acquired volume pulse wave.

The camera 101 captures an image of the skin of the living body LB, preferably an image of the skin of the face of the living body LB. If the camera 101 captures an image of the skin of the face of the living body LB, the volume pulse wave can be acquired from a signal that is in accordance with the light reflected off the skin which has a large area and which includes many blood vessels therebelow. Therefore, the volume pulse wave can be easily acquired.

### 1.2 Camera

FIG. 2 is a schematic cross-sectional view of a camera included in the measuring instrument in accordance with Embodiment 1.

Referring to FIG. 2, the camera 101 includes a lens 111, an imaging element 112, and a support member 113.

The lens 111, the imaging element 112, and the support member 113 are integrated.

The lens 111 guides the light reflected off the living body LB to the imaging element 112. The lens 111 focuses the reflected light onto the imaging element 112 to form an image of the living body LB on the imaging element 112.

Referring to FIG. 2, the imaging element 112 includes a plurality of pixels 121. The plurality of pixels 121 are arranged in a matrix in a plane that is perpendicular to the optical axis of the lens 111. The imaging element 112 outputs a plurality of signals respectively representing the quantities of the light received by the plurality of pixels 121. The plurality of outputted signals represent an image. The imaging element 112 is a complementary metal oxide semiconductor image sensor (CIS). The imaging element 112 may be a non-CIS image sensor. For example, the imaging element 112 may be a charge coupled device (CCD) image sensor.

The support member 113 supports the lens 111.

### 1.3 First Peak Sensitivity Wavelengths of First Pixels and Second Peak Sensitivity Wavelength of Second Pixels

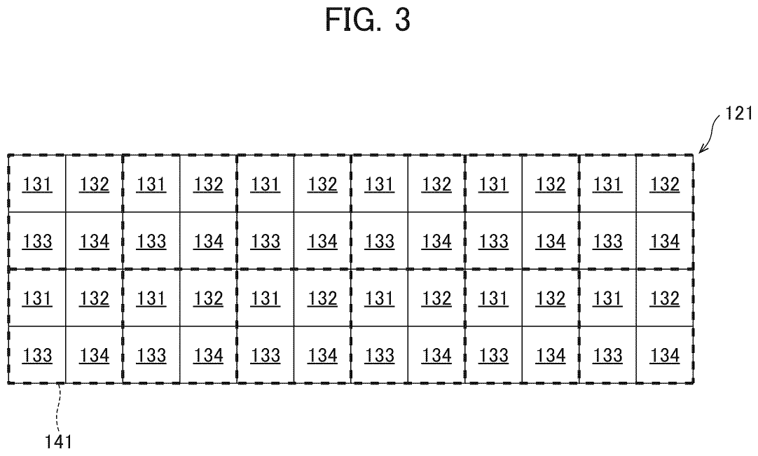

FIG. 3 is a schematic plan view of a plurality of pixels included in the measuring instrument in accordance with Embodiment 1.

Referring to FIG. 3, the plurality of pixels 121 include three types of first pixels 131, 132, and 133 and one type of second pixels 134. The first pixels 131, 132, and 133 and the second pixels 134 may be arranged differently from the arrangement shown in FIG. 3. In particular, the positions of the second pixels 134 may differ from those positions of the second pixels 134 which are shown in FIG. 3.

The first pixels 131, 132, and 133 are used to acquire signals representing the quantities of the received light that has wavelength components that are not easily affected by the oxygen saturation level of the living body LB. Therefore, the first pixels 131, 132, and 133 have first peak sensitivity wavelengths that are specified so that the absorption coefficient of oxidized hemoglobin to the light that has the first peak sensitivity wavelengths does not differ significantly from the absorption coefficient of reduced hemoglobin to the light that has the same peak sensitivity wavelengths. Therefore, the first peak sensitivity wavelengths of the first pixels 131, 132, and 133 are specified to be either less than or equal to 600 nm or greater than or equal to 760 nm. The first peak sensitivity wavelengths of the first pixels 131, 132, and 133 differ from each other.

The plurality of pixels 121 may include two types of first pixels and may include four or more types of first pixels.

The second pixels 134 are used to acquire signals representing the quantities of the received light that has wavelength components that are easily affected by the oxygen saturation level of the living body LB. Therefore, the second pixels 134 have a second peak sensitivity wavelength that is specified so that the absorption coefficient of oxidized hemoglobin to the light that has the second peak sensitivity wavelength does not differ significantly from the absorption coefficient of reduced hemoglobin to the light that has the same peak sensitivity wavelength. Therefore, the second peak sensitivity wavelength of the second pixels 134 is specified to be greater than or equal to 620 nm and less than or equal to 740 nm.

The plurality of pixels 121 may include two or more types of second pixels.

The peak sensitivity wavelength of a pixel refers to the wavelength at which the sensitivity has a peak in the spectral sensitivity of the pixel.

The imaging element 112 includes a plurality of pixel blocks 141. The plurality of pixel blocks 141 are arranged in a matrix in a plane that is perpendicular to the optical axis of the lens 111. Each pixel block 141 includes one first pixel 131, one first pixel 132, one first pixel 133, and one second pixel 134. In each pixel block 141, the first pixel 131, the first pixel 132, the first pixel 133, and the second pixel 134 are arranged in a matrix. The first pixel 131, the first pixel 132, the first pixel 133, and the second pixel 134 are arranged in the same manner across the plurality of pixel blocks 141.

### 1.4 Spectral Sensitivity

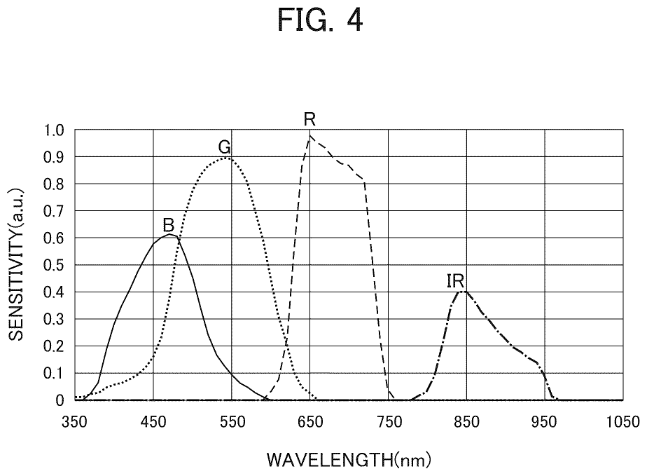

FIG. 4 is a graph representing an example of the spectral sensitivities of pixels included in a biological information measuring instrument in accordance with Embodiment 1.

FIG. 4 shows wavelengths on the horizontal axis and sensitivities on the vertical axis. In the example shown in FIG. 4, the first pixel 131 is a green pixel that exhibits high sensitivity to green (G) light and has a first peak sensitivity wavelength of approximately 540 nm, which falls in the range of less than or equal to 600 nm. In addition, the first pixel 132 is a blue pixel that exhibits high sensitivity to blue (B) light and has a first peak sensitivity wavelength of approximately 470 nm, which falls in the range of less than or equal to 600 nm. In addition, the first pixel 133 is an infrared light pixel that exhibits high sensitivity to infrared (IR) light and has a first peak sensitivity wavelength of approximately 850 nm, which falls in the range of greater than or equal to 760 nm.

In the example shown in FIG. 4, the three first peak sensitivity wavelengths include two peak sensitivity wavelengths that fall in the range of less than or equal to 600 nm and one peak sensitivity wavelength that falls in the range of greater than or equal to 760 nm. The three first peak sensitivity wavelengths may include three peak sensitivity wavelengths that fall in the range of less than or equal to 600 nm, may include one peak sensitivity wavelength that falls in the range of less than or equal to 600 nm and two peak sensitivity wavelengths that fall in the range of greater than or equal to 760 nm, and may include three peak sensitivity wavelengths that fall in the range of greater than or equal to 760 nm.

The absorption coefficient of oxidized hemoglobin to the light that has a wavelength of 420 nm differs slightly from the absorption coefficient of reduced hemoglobin to the light that has the same wavelength. The absorption coefficient of oxidized hemoglobin to the light that has a wavelength of 550 nm differs slightly from the absorption coefficient of reduced hemoglobin to the light that has the same wavelength. However, when the first pixels 131, 132, and 133 exhibit the spectral sensitivities shown in FIG. 4, the influence is negligible of the difference between the absorption coefficient of oxidized hemoglobin to the light that has a wavelength of 420 nm and the absorption coefficient of reduced hemoglobin to the light that has the same wavelength and of the difference between the absorption coefficient of oxidized hemoglobin to the light that has a wavelength of 550 nm and the absorption coefficient of reduced hemoglobin to the light that has the same wavelength.

In addition, the absorption coefficient of oxidized hemoglobin to the light that has a wavelength of greater than or equal to 760 nm differs slightly from the absorption coefficient of reduced hemoglobin to the light that has the same wavelength. However, when the first pixels 131, 132, and 133 exhibit the spectral sensitivities shown in FIG. 4, the influence is negligible of the difference between the absorption coefficient of oxidized hemoglobin to the light that has a wavelength of greater than or equal to 760 nm and the absorption coefficient of reduced hemoglobin to the light that has the same wavelength. This is because the difference between the absorption coefficient of oxidized hemoglobin to the light that has a wavelength of greater than or equal to 760 nm and the absorption coefficient of reduced hemoglobin to the light that has the same wavelength is smaller than the difference between the absorption coefficient of oxidized hemoglobin to the light that has a wavelength of greater than or equal to 620 nm and less than or equal to 740 nm and the absorption coefficient of reduced hemoglobin to the light that has the same wavelength.

In the example shown in FIG. 4, the second pixel 134 is a red pixel that exhibits high sensitivity to red (R) light and has a second peak sensitivity wavelength of approximately 650 nm that falls in the range of greater than or equal to 600 nm and less than or equal to 740 nm.

### 1.5 Calculating Oxygen Saturation

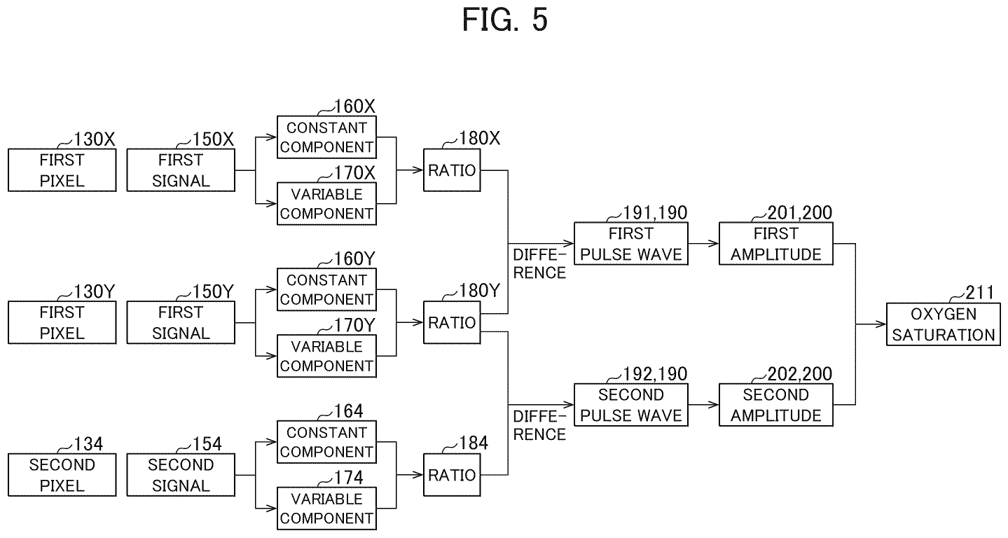

FIG. 5 is a diagram illustrating an oxygen saturation level calculation process performed by a processing unit included in the measuring instrument in accordance with Embodiment 1.

Referring to FIG. 5, the processing unit 102 acquires two first signals 150X and 150Y and one second signal 154. The acquired first signals 150X and 150Y respectively represent the quantities of the light received by two types of first pixels 130X and 130Y that are among the first pixels 131, 132, and 133. The acquired second signal 154 represents the quantity of the light received by one type of second pixel 134.

The first signals 150X and 150Y and the second signal 154 contain a volume pulse wave. However, the first signals 150X and 150Y and the second signal 154 are easily affected by, for example, body movements of the living body LB and variations of environmental light. The first signals 150X and 150Y are not easily affected by the oxygen saturation level of the living body LB and are used as reference signals. The second signal 154 is easily affected by the oxygen saturation level of the living body LB.

The first signal 150X may be either one first signal representing the quantity of the light received by one first pixel 130X or an average of two or more first signals respectively representing the quantities of the light received by two or more first pixels 130X. When the first signal 150X is an average of two or more first signals, the imaging element 112 performs a signal process of computing an average of two or more first signals and outputs the computed average, and the processing unit 102 acquires the outputted average. Alternatively, the imaging element 112 outputs two or more first signals, a system that excludes the imaging element 112 and the processing unit 102 performs a signal process of computing an average of the two or more outputted first signals and outputs the computed average, and the processing unit 102 acquires the outputted average. As another alternative, the imaging element 112 outputs two or more first signals, and the processing unit 102 performs a signal process of computing an average of the two or more outputted first signals, to acquire an average of the two or more first signals. When the first signal 150X is an average of two or more first signals, the first signal 150X can have an improved signal-to-noise ratio. The first signal 150X represents temporal changes of either a signal value or a signal quantity that represents the quantity of the light received by one first pixel 130X.

Likewise, the first signal 150Y may be either one first signal representing the quantity of the light received by one first pixel 130Y or an average of two or more first signals respectively representing the quantities of the light received by two or more first pixels 130Y. When the first signal 150Y is an average of two or more first signals, the imaging element 112 performs a signal process of computing an average of two or more first signals and outputs the computed average, and the processing unit 102 acquires the outputted average. Alternatively, the imaging element 112 outputs two or more first signals, a system that excludes the imaging element 112 and the processing unit 102 performs a signal process of computing an average of the two or more outputted first signals and outputs the computed average, and the processing unit 102 acquires the outputted average. As another alternative, the imaging element 112 outputs two or more first signals, and the processing unit 102 performs a signal process of computing an average of the two or more outputted first signals, to acquire an average of the two or more first signals. When the first signal 150Y is an average of two or more first signals, the first signal 150Y can have an improved signal-to-noise ratio. The first signal 150Y represents temporal changes of either a signal value or a signal quantity that represents the quantity of the light received by the first pixel 130X.

Likewise, the second signal 154 may be either one second signal representing the quantity of the light received by one second pixel 134 or an average of two or more second signals respectively representing the quantities of the light received by two or more second pixels 134. When the second signal 154 is an average of two or more second signals, the imaging element 112 performs a signal process of computing an average of two or more second signals and outputs the computed average, and the processing unit 102 acquires the outputted average. Alternatively, the imaging element 112 outputs two or more second signals, a system that excludes the imaging element 112 and the processing unit 102 performs a signal process of computing an average of the two or more outputted second signals and outputs the computed average, and the processing unit 102 acquires the outputted average. As another alternative, the imaging element 112 outputs two or more second signals, and the processing unit 102 performs a signal process of computing an average of the two or more outputted second signals, to acquire an average of the two or more second signals. When the second signal 154 is an average of two or more second signals, the second signal 154 can have an improved signal-to-noise ratio. The second signal 154 represents temporal changes of either a signal value or a signal quantity that represents the quantity of the light received by the second pixel 134.

The processing unit 102 acquires a first pulse wave 191 from the two first signals 150X and 150Y. The first signals 150X and 150Y are not easily affected by the oxygen saturation level of the living body LB. Therefore, the first pulse wave 191, which is acquired from the first signals 150X and 150Y, is not easily affected by the oxygen saturation level of the living body LB. The processing unit 102 may acquire the first pulse wave 191 from three or more first signals.

The processing unit 102, in acquiring the first pulse wave 191 from the two first signals 150X and 150Y, separates each first signal included in the two first signals 150X and 150Y into a constant component and a variable component mutually, to acquire a ratio from the mutually separated constant and variable components. In other words, the processing unit 102 separates the first signal 150X into a constant component 160X and a variable component 170X mutually to acquire a ratio 180X from the mutually separated constant and variable components 160X and 170X and separates the first signal 150Y into a constant component 160Y and a variable component 170Y mutually to acquire a ratio 180Y from the mutually separated constant and variable components 160Y and 170Y. The constant components 160X and 160Y can be safely assumed to be invariable to the heart rate over a sufficiently long, certain period of time. The variable components 170X and 170Y are variable with passage of time. The ratio 180X is calculated by dividing one of the constant component 160X and the variable component 170X by the other one of the constant component 160X and the variable component 170X. The ratio 180Y is calculated by dividing one of the constant component 160Y and the variable component 170Y by the other one of the constant component 160Y and the variable component 170Y.

The processing unit 102 acquires the first pulse wave 191 by calculating a difference between the two ratios 180X and 180Y acquired respectively for the two first signals 150X and 150Y.

The variations of the quantity of light that has wavelength components contained in the light received by the pixel caused by, for example, body movements of the living body LB and variations of environmental light hardly depend on the wavelengths of these wavelength components. Therefore, the ratio of the constant component and the variable component acquired from a signal representing the quantity of the light received by the pixel hardly depends on the peak sensitivity wavelength of the pixel. Therefore, the first pulse wave 191 that is not easily affected by, for example, body movements of the living body LB and variations of environmental light can be acquired by acquiring the first pulse wave 191 by calculating a difference between the ratios 180X and 180Y acquired respectively for the first signals 150X and 150Y. This particular approach enables acquiring the first pulse wave 191 that primarily reflects the difference between the first signals 150X and 150Y that is caused by the mutual difference between the first peak sensitivity wavelengths of the first pixels 130X and 130Y.

When the first pixels 130X and 130Y are green and blue pixels, the first pulse wave 191 is calculated by equation (1) below.

$$First Pulse Wave = Signal from Green Pixel - Signal from Blue Pixel \qquad ( 1 )$$

The signal from a green pixel in equation (1) is a ratio acquired from a constant component and a variable component contained in a first signal representing the quantity of the light received by the green pixel. The signal from a blue pixel in equation (1) is a ratio acquired from a constant component and a variable component contained in a first signal representing the quantity of the light received by the blue pixel.

The processing unit 102 acquires a second pulse wave 192 from the first signal 150Y and the second signal 154.

The processing unit 102, in acquiring the second pulse wave 192 from the first signal 150Y and the second signal 154, separates each signal included in the first signal 150Y and the second signal 154 into a constant component and a variable component mutually, to acquire a ratio from the mutually separated constant and variable components. In other words, the processing unit 102 separates the first signal 150Y into the constant component 160Y and the variable component 170Y mutually, to acquire the ratio 180Y from the mutually separated constant and variable components 160Y and 170Y, and separates the second signal 154 into a constant component 164 and a variable component 174 mutually, to acquire a ratio 184 from the mutually separated constant and variable components 164 and 174. The constant components 160Y and 164 can be safely assumed to be invariable to the heart rate over a sufficiently long, certain period of time. The variable components 170Y and 174 are variable with passage of time. The ratio 180Y is calculated by dividing one of the constant component 160Y and the variable component 170Y by the other one of the constant component 160Y and the variable component 170Y. The ratio 184 is calculated by dividing one of the constant component 164 and the variable component 174 by the other one of the constant component 164 and the variable component 174.

The processing unit 102 acquires the second pulse wave 192 by calculating a difference between the two ratios 180Y and 184 acquired respectively for the first signal 150Y and the second signal 154.

The variations of the quantity of light that has wavelength components contained in the light received by the pixel caused by, for example, body movements of the living body LB and variations of environmental light hardly depend on the wavelengths of these wavelength components. Therefore, the ratio of the constant component and the variable component acquired from a signal representing the quantity of the light received by the pixel hardly depends on the peak sensitivity wavelength of the pixel. Therefore, the second pulse wave 192 that is not easily affected by, for example, body movements of the living body LB and variations of environmental light can be acquired by acquiring the second pulse wave 192 by calculating a difference between the two ratios 180Y and 184 acquired respectively for the first signal 150Y and the second signal 154. This particular approach enables acquiring the second pulse wave 192 that primarily reflects the difference between the first signal 150Y and the second signal 154 that is caused by the mutual difference between the first peak sensitivity wavelength of the first pixel 130Y and the second peak sensitivity wavelength of the second pixel 134.

When the first pixel 130Y is a green pixel, and the second pixel 134 is a red pixel, the second pulse wave 192 is calculated by equation (2) below.

Second Pulse Wave=Signal from Green Pixel−Signal from Red Pixel (2)

The signal from a green pixel in equation (2) is a ratio acquired from a constant component and a variable component contained in a first signal representing the quantity of the light received by the green pixel. The signal from a red pixel in equation (2) is a ratio acquired from a constant component and a variable component contained in a second signal representing the quantity of the light received by the red pixel.

The processing unit 102 acquires a first amplitude 201 of the first pulse wave 191 and a second amplitude 202 the second pulse wave 192. For example, the processing unit 102 acquires a peak value of the first pulse wave 191 to acquire the first amplitude 201 from the acquired peak value and acquires a peak value of the second pulse wave 192 to acquire the second amplitude 202 from the acquired peak value. The processing unit 102 may acquire a plurality of peak values of the first pulse wave 191 to acquire the first amplitude 201 from an average value of the plurality of acquired peak values and may acquire a plurality of peak values of the second pulse wave 192 to acquire the second amplitude 202 from an average value of the plurality of acquired peak values.

The acquired first amplitude 201 hardly depends on the presence ratio of oxidized hemoglobin and reduced hemoglobin. The acquired second amplitude 202 depends on this presence ratio. By taking advantage of this fact, the processing unit 102 acquires an oxygen saturation level 211 from the acquired first and second amplitudes 201 and 202. For example, the processing unit 102 acquires a value by dividing the second amplitude 202 by the first amplitude 201 to acquire the oxygen saturation level 211 from the acquired value. The processing unit 102 refers to a table that associates a plurality of values respectively with a plurality of oxygen saturation levels and specifies the oxygen saturation level associated in this table with the acquired value as the acquired oxygen saturation level 211. This table is prepared in advance on the basis of measurements by a system that excludes the measuring instrument 1. The processing unit 102 may convert, to an oxygen saturation level, a value acquired in reference to a conversion formula for converting a value to an oxygen saturation level, to specify this oxygen saturation level as the acquired oxygen saturation level 211.

As described here, the oxygen saturation level 211 of the living body LB can be measured with high precision in a contactless manner without blocking environmental light, by acquiring a plurality of pulse waves including the first pulse wave 191 and the second pulse wave 192 from the first signals 150X and 150Y and the second signal 154 and acquiring the oxygen saturation level 211 from the plurality of acquired pulse waves.

### 1.6 Calculating Amplitude of Pulse Wave

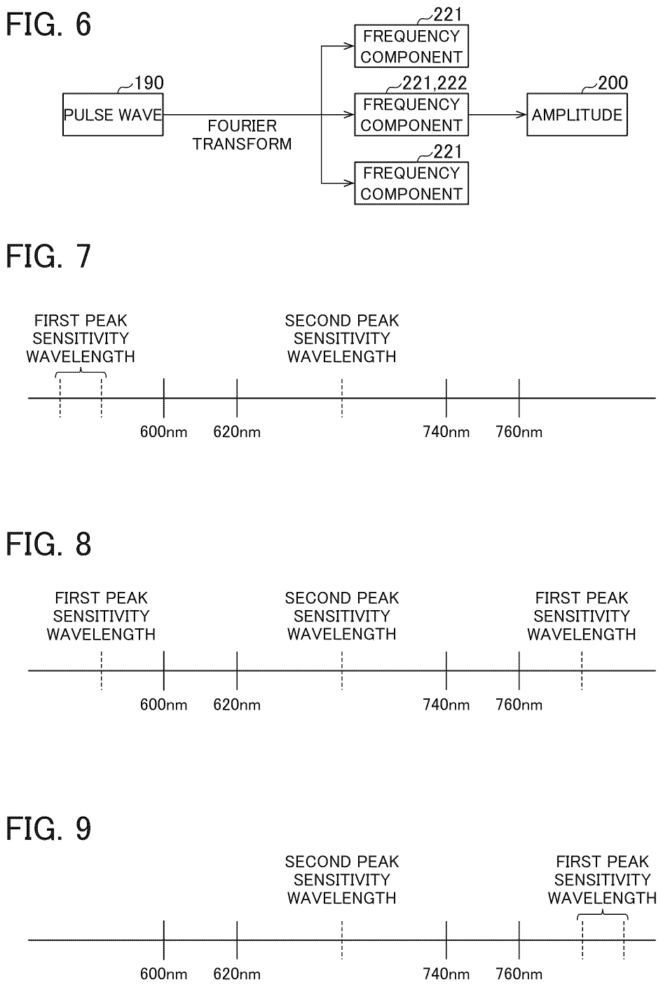

FIG. 6 is a diagram illustrating a pulse wave amplitude calculation process performed by the processing unit included in the measuring instrument in accordance with Embodiment 1.

Referring to FIG. 6, the processing unit 102, in calculating an amplitude 200 of each pulse wave 190 included in the first pulse wave 191 and the second pulse wave 192, preferably acquires a plurality of frequency components 221 contained in each pulse wave 190 by performing Fourier transform on each pulse wave 190.

In addition, the processing unit 102 selects a frequency component 222 that has a particular frequency from the plurality of acquired frequency components 221 to acquire the amplitude 200 of each pulse wave 190 from the selected frequency component 222. For example, the processing unit 102 acquires a peak value of the selected frequency component 222 to acquire the amplitude 200 of each pulse wave 190 from the acquired peak value. The particular frequency is, for example, a frequency that corresponds to the frequency of the heart rate.

### 1.7 Examples of First Peak Sensitivity Wavelengths

FIGS. 7, 8, and 9 are diagrams illustrating examples of the peak sensitivity wavelengths of pixels included in the measuring instrument in accordance with Embodiment 1.

In the example shown in FIG. 7, the two types of first pixels 130X and 130Y are two types of visible pixels that exhibit high sensitivity to visible light. The two types of visible pixels are, for example, a green pixel that exhibits high sensitivity to green light and a blue pixel that exhibits high sensitivity to blue light. Therefore, the two types of first pixels 130X and 130Y exhibit two first peak sensitivity wavelengths that are two peak sensitivity wavelengths of less than or equal to 600 nm. In this example, when there are included three or more types of first pixels, the three or more types of first pixels exhibit three or more first peak sensitivity wavelengths that are three or more peak sensitivity wavelengths of less than or equal to 600 nm.

In the example shown in FIG. 8, the two types of first pixels 130X and 130Y are one type of visible pixel that exhibits high sensitivity to visible light and one type of infrared light pixel that exhibits high sensitivity to infrared light. The one type of visible pixel is, for example, either a green pixel that exhibits high sensitivity to green light or a blue pixel that exhibits high sensitivity to blue light. Therefore, the two types of first pixels 130X and 130Y exhibit two first peak sensitivity wavelengths that are one peak sensitivity wavelength of less than or equal to 600 nm and one peak sensitivity wavelength of greater than or equal to 760 nm. In this example, when there are included three or more types of first pixels, the three or more types of first pixels exhibit three or more first peak sensitivity wavelengths that are one or more peak sensitivity wavelengths of less than or equal to 600 nm and one or more peak sensitivity wavelengths of greater than or equal to 760 nm. The one or more peak sensitivity wavelengths of less than or equal to 600 nm may be two or more peak sensitivity wavelengths, and the one or more peak sensitivity wavelengths of greater than or equal to 760 nm may be two or more peak sensitivity wavelengths.

In the example shown in FIG. 9, the two types of first pixels 130X and 130Y are two types of infrared light pixels that exhibit high sensitivity to infrared light. Therefore, the two types of first pixels 130X and 130Y exhibit two first peak sensitivity wavelengths that are two peak sensitivity wavelengths of greater than or equal to 760 nm. In this example, when there are included three or more types of first pixels, the three or more types of first pixels exhibit three or more first peak sensitivity wavelengths that are three or more peak sensitivity wavelengths of greater than or equal to 760 nm.

### 1.8 Flow of Process

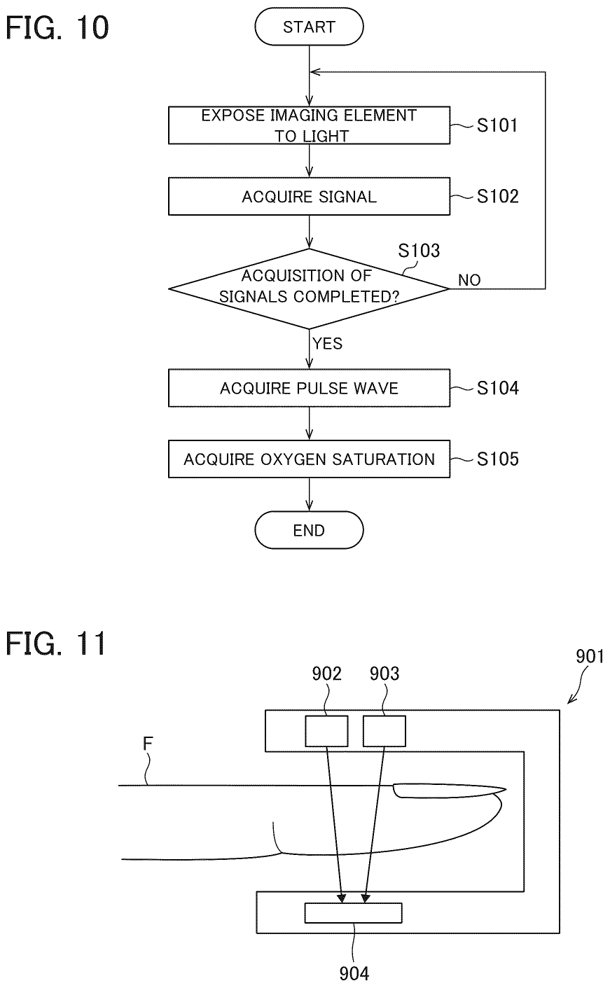

FIG. 10 is a flow chart representing a flow of a process performed by the processing unit included in the measuring instrument in accordance with Embodiment 1.

The processing unit 102 implements steps S101 to S105 shown in FIG. 10.

In step S101, the processing unit 102 causes the imaging element 112 to be exposed to light to cause the imaging element 112 to output a signal.

In immediately succeeding step S102, the processing unit 102 acquires the signal outputted as a result of the exposure to light.

In immediately succeeding step S103, the processing unit 102 determines whether or not the acquisition of the first signal 150X, the first signal 150Y, and the second signal 154 configured by signals acquired so far is completed. If the processing unit 102 has determined that the acquisition of the first signal 150X, the first signal 150Y, and the second signal 154 is completed, the processing unit 102 implements step S104. If the processing unit 102 has determined that the acquisition of the first signal 150X, the first signal 150Y, and the second signal 154 is not completed, the processing unit 102 implements step S101.

In steps S101 to S103, the processing unit 102 completes the acquisition of the first signal 150X, the first signal 150Y, and the second signal 154 by repeatedly implementing steps S101 and S102 until the acquisition of the first signal 150X, the first signal 150Y, and the second signal 154 is completed.

In step S104, the processing unit 102 acquires the first pulse wave 191 and the second pulse wave 192 from the first signal 150X, the first signal 150Y, and the second signal 154 which have been acquired.

In step S105, the processing unit 102 acquires the oxygen saturation level 211 from the first pulse wave 191 and the second pulse wave 192 which have been acquired.

### 1.9 Comparing with Pulse Oximeter

FIG. 11 is a schematic illustration of a pulse oximeter for comparison with the measuring instrument in accordance with Embodiment 1.

To measure an oxygen saturation level with a pulse oximeter 901 shown in FIG. 11, one places a finger F into contact with the pulse oximeter 901. In addition, the red light emitted by a first light-emitting element 902 and the infrared light emitted by a second light-emitting element 903 are alternately projected onto the finger F. In addition, the red and the infrared light transmitted by the finger F is received by a light-receiving element 904. In addition, computation is done on the output of the light-receiving element 904 to calculate an oxygen saturation level. To measure an oxygen saturation level with the pulse oximeter 901, the influence of, for example, body movements of the living body and variations of environmental light can be restrained by placing the finger F into contact with the pulse oximeter 901. However, to measure the oxygen saturation level with the pulse oximeter 901, the finger F needs to be fixed to regulate the movement of the living body. In addition, it becomes difficult to continuously measure the oxygen saturation level.

In contrast, the measuring instrument 1 in accordance with Embodiment 1 is capable of measuring the oxygen saturation level 211 in a contactless manner without having to regulate the movement of the living body LB. In addition, the measuring instrument 1 in accordance with Embodiment 1 is also capable of continuously measuring the oxygen saturation level 211.

2 Embodiment 2

The following will describe differences of Embodiment 2 from Embodiment 1. The description may be silent about the structures and features of Embodiment 2 that are the same as those of Embodiment 1.

FIG. 12 is a block diagram of a measuring instrument in accordance with Embodiment 2.

A measuring instrument 2 in accordance with Embodiment 2 shown in FIG. 12 includes a light-emitting element 231.

The light-emitting element 231 emits light L to project the emitted light L onto the living body LB. The emitted light L contains wavelength components that have wavelengths that fall respectively in a first sensitivity wavelength range of the first pixel 130X, a first sensitivity wavelength range of the first pixel 130Y, and a second sensitivity wavelength range of the second pixel 134.

The sensitivity wavelength range of a pixel refers to the wavelength range in which the pixel has sensitivity.

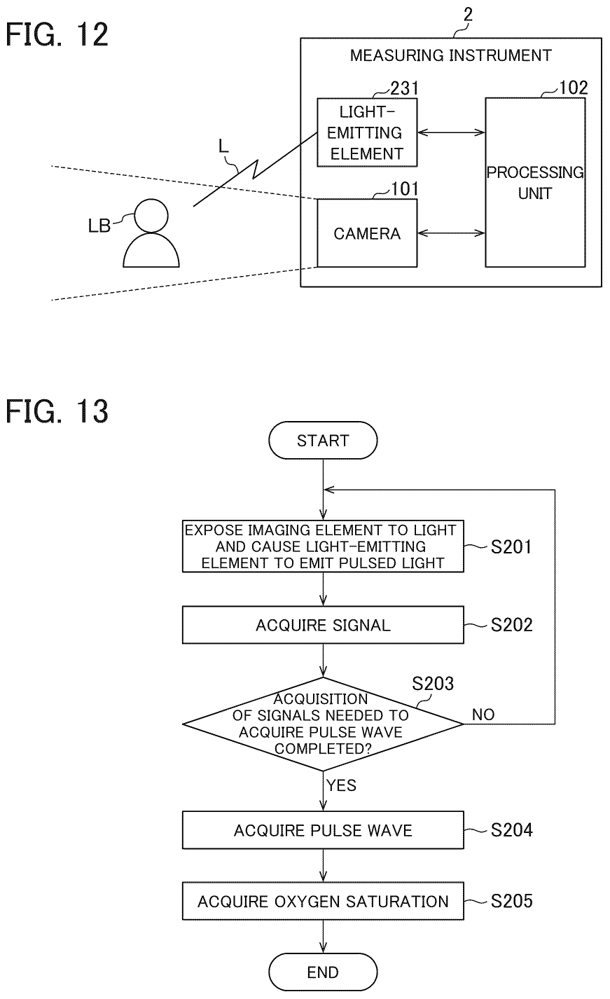

FIG. 13 is a flow chart representing a flow of a process performed by a processing unit included in the measuring instrument in accordance with Embodiment 2.

The processing unit 102 implements steps S201 to S205 shown in FIG. 13.

In step S201, the processing unit 102 causes the imaging element 112 to be exposed to light. In addition, the processing unit 102 causes the light-emitting element 231 to turn on and off by causing the light-emitting element 231 to emit pulsed light in synchronism with the exposure timings at which the processing unit 102 causes the imaging element 112 to be exposed to light.

In the immediately succeeding steps S202 to S205, the processing unit 102 respectively implements the same process as the processing unit 102 implements in steps S102 to S105 shown in FIG. 10.

This particular approach enables measuring the oxygen saturation level 211, for example, when there are large variations of environmental light and when there is no environmental light.

The processing unit 102 may cause the light-emitting element 231 to continuously emit light while repeatedly implementing steps S201 to S203.

3 Embodiment 3

The following will describe differences of Embodiment 3 from Embodiment 1. The description may be silent about the structures and features of Embodiment 3 that are the same as those of Embodiment 1.

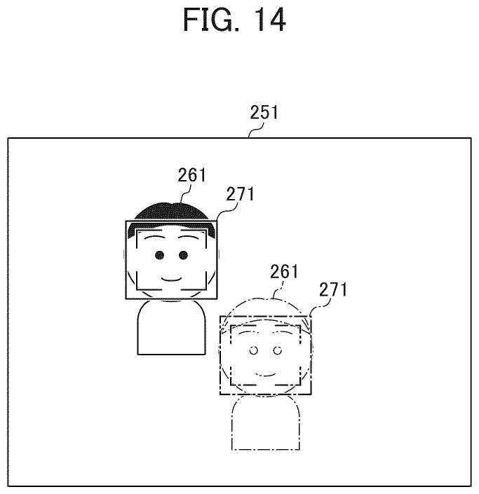

FIG. 14 is an illustration of images each represented by a plurality of signals outputted by an imaging element included in a measuring instrument in accordance with Embodiment 3 and also of frames each indicating an area specified by a processing unit included in this measuring instrument.

Referring to FIG. 14, in the measuring instrument in accordance with Embodiment 3, the processing unit 102 moves the area used to calculate the oxygen saturation level 211 so that the area follows the movement of an image 261 of the living body LB in an image 251 represented by a plurality of signals outputted by the imaging element 112, as indicated by the movement of a frame 271. The first pixel 130X, the first pixel 130Y, and the second pixel 134 used in the calculation of the oxygen saturation level 211 are pixels that belong to this area. This particular approach enables maintaining, even in the presence of body movements of the living body LB, a state of the living body LB where the oxygen saturation level 211 is acquired from signals that are suited to the acquisition of the oxygen saturation level 211 and that are in accordance with the light reflected off the body surface. For example, a state can be maintained where the oxygen saturation level 211 is acquired from signals that are in accordance with the light reflected off a cheek of the living body LB.

The measuring instrument 1 in accordance with Embodiment 1 is capable of measuring the oxygen saturation level 211 while restraining the influence of body movements of the living body LB if the body movements of the living body LB are small. However, the measuring instrument 1 in accordance with Embodiment 1 may not be capable of measuring the oxygen saturation level 211 while restraining the influence of body movements of the living body LB if the body movements of the living body LB are large.

In contrast, the measuring instrument in accordance with Embodiment 3 is capable of measuring the oxygen saturation level 211 while restraining the influence of body movements of the living body LB even if the body movements of the living body LB are large.

While there have been described what are at present considered to be certain embodiments of the invention, it will be understood that various modifications may be made thereto, and it is intended that the appended claims cover all such modifications as fall within the true spirit and scope of the invention.

What is claimed is:

1. A measuring instrument comprising:
a camera that captures an image of a living body at a distance from the living body, the camera comprising an imaging element comprising:
a plurality of types of first pixels that respectively exhibit a plurality of mutually different first peak sensitivity wavelengths of either less than or equal to 600 nm or greater than or equal to 760 nm; and
a second pixel that exhibits a second peak sensitivity wavelength of greater than or equal to 620 nm and less than or equal to 740 nm; and
a processing unit that acquires a plurality of pulse waves from a plurality of first signals respectively representing quantities of light received by the plurality of types of first pixels and also from a second signal representing a quantity of light received by the second pixel and that acquires an oxygen saturation level of the living body from the plurality of pulse waves.

2. The measuring instrument according to claim 1, wherein
the processing unit acquires a first pulse wave from the plurality of first signals and acquires a second pulse wave from the second signal and a first signal included in the plurality of first signals, and
the plurality of pulse waves include the first pulse wave and the second pulse wave.

3. The measuring instrument according to claim 1, wherein the plurality of first peak sensitivity wavelengths are less than or equal to 600 nm.

4. The measuring instrument according to claim 1, wherein the plurality of first peak sensitivity wavelengths include a peak sensitivity wavelength of less than or equal to 600 nm and a peak sensitivity wavelength of greater than or equal to 760 nm.

5. The measuring instrument according to claim 1, wherein the plurality of first peak sensitivity wavelengths are greater than or equal to 760 nm.

6. The measuring instrument according to claim 1, wherein
the plurality of types of first pixels have a plurality of first sensitivity wavelength ranges respectively,
the second pixel has a second sensitivity wavelength range,
the measuring instrument further comprises a light-emitting element that emits light including wavelength components that have wavelengths that respectively fall in the plurality of first sensitivity wavelength ranges and the second sensitivity wavelength range, and
the processing unit causes the light-emitting element to emit pulsed light in synchronism with an exposure timing at which the processing unit causes the imaging element to be exposed to light.

7. The measuring instrument according to claim 1, wherein
the imaging element includes a plurality of pixels, and
the processing unit moves an area to which the plurality of types of first pixels and the second pixel belong so that the area follows movement of the image of the living body in an image represented by a plurality of signals respectively representing quantities of light received by the plurality of pixels.

8. The measuring instrument according to claim 2, wherein the processing unit
separates each of the first signals included in the plurality of first signals into a constant component and a variable component mutually, to acquire a ratio from the constant component and the variable component, and
calculates a difference between a plurality of ratios acquired respectively for the plurality of first signals to acquire the first pulse wave.

9. The measuring instrument according to claim 2, wherein the processing unit separates each signal included in the first signal and the second signal into a constant component and a variable component mutually, acquires a ratio from the constant component and the variable component, and calculates a difference between two ratios acquired respectively for the first signal and the second signal to acquire the second pulse wave.

10. The measuring instrument according to claim 1, wherein the processing unit acquires amplitudes of the plurality of pulse waves to acquire the oxygen saturation level from the amplitudes of the plurality of pulse waves.

11. The measuring instrument according to claim 10, wherein the processing unit selects a frequency component that has a particular frequency from a plurality of frequency components contained in each pulse wave included in the plurality of pulse waves to acquire an amplitude of that pulse wave from the frequency component.
